(19) United States
(12) Patent Application Publication
(10) Pub. No.: US 2024/0398690 A1
(43) Pub. Date: Dec. 5, 2024

(54) Herbal Cleansing Shampoo

(71) Applicant: AIH PERSONAL CARE PRO, LLC, Schaumburg, IL (US)

(72) Inventors: Abdessettar Ibourki, Schaumburg, IL (US); Sharon Martiny, Alton (GB); Khadija Ibourki, Khouribga (MA)

(73) Assignee: AIH PERSONAL CARE PRO, LLC, Schaumburg, IL (US)

(21) Appl. No.: 18/619,896

(22) Filed: Mar. 28, 2024

Related U.S. Application Data
(60) Provisional application No. 63/469,864, filed on May 31, 2023.

(51) Int. Cl. A61K 8/9789; A61K 8/34; A61K 8/92; A61Q 5/02

(52) U.S. Cl. CPC A61K 8/9789; A61K 8/345; A61K 8/922; A61Q 5/02; A61K 2800/5922; A61K 2800/596

(57) ABSTRACT

An herbal cleansing shampoo is provided that includes an aqueous base, an herbal extract blend of natural herbal active ingredients, a surfactant blend component, and a natural oil component. The components work together to clean and moisturize dry, brittle hair and contribute to a shiny, luscious appearance. The herbal cleansing shampoo can be used alone or in combination with an all-natural argan oil blend hair treatment and an all-natural rinse for optimal results.

### RELATED APPLICATIONS

This patent application claims priority to U.S. Provisional Application 63/469,864, filed on May 31, 2023, the disclosure of which is incorporated herein by reference.

### FIELD OF THE INVENTION

This invention is directed to an herbal cleansing shampoo formed using natural herbal active ingredients. The herbal cleansing shampoo can be applied alone or in sequence with an all-natural conditioner hair treatment and an all-natural rinse to help eliminate dry, brittle hair and replace it with healthy hair and luscious locks.

### BACKGROUND OF THE INVENTION

Hair treatment products have been developed over the years with the objective of providing cleaner, shinier, and healthier looking hair. Many of these products contain synthetic ingredients that are effective in shampooing and cleaning the hair, but which can result in dry scalp, dandruff and dry or brittle hair Additionally, the widespread use of synthetic ingredients can result in contamination of rivers, streams, and groundwater, causing harmful effects to fish and other wildlife. There is a need or desire for hair treatment formulas (including shampoos) that are not only effective in producing cleaner and shinier hair, but also avoid damage to the users' hair and scalp, and which do not cause harm to the surrounding environment.

### SUMMARY OF THE INVENTION

This invention is directed to an herbal cleansing shampoo that can be applied alone or in sequence with other all-natural conditioner and/or rinse. The herbal cleansing shampoo contains all natural active ingredients that promote healthy, shiny hair with luscious locks and have environmental advantages.

The herbal cleansing shampoo can include some or all of the following ingredients, which can be present in the amounts described herein.

Water Base: preferably deionized water.

Herbal Extracts: ocmium basilicum leaf extract, citrus aurantium amara flower extract, rosmarius officinalis leaf extract, chamomilia recutia flower extract, lavandula angustifolia flower extract, artemisia vulgaris herb extract, ocmium basilicum seed extract, eugenia caryophyllus bud extract, thymus vulgaris extract.

Surfactants: guar hydroxypropyltrimonium chloride, aqueous cocamidopropyl betaine, aqueous disodium laureth suphosuccinate, sodium methyl cocoyl taurate, sodium cocoyl isethionate.

Natural Oil: glycerin, tea tree oil.

Performance and processing aids: citric acid, centrimonium chloride, sodium benzoate, potassium sorbate, pentylene glycol, sodium anisate, aqueous trisodium ethylenediamine disuccinate, hydroxypropyl methylcellulose.

In one embodiment, it is a feature and advantage of the invention to provide an herbal cleansing shampoo that includes:
about 40% to about 70% by weight water;
about 2% to about 20% by weight of an herbal extract blend component;
about 10% to about 40% by weight of a surfactant blend component; and
about 1% to about 15% by weight of a natural oil component;
wherein the herbal extract component includes three or more of ocmium basilicum leaf extract, citrus aurantium amara flower extract, rosmarius officinalis leaf extract, chamomilia recutia flower extract, lavandula angustifolia flower extract, artemisia vulgaris herb extract, ocmium basilicum seed extract, eugenia caryophyllus bud extract, and thymus vulgaris extract.

In another embodiment, it is a feature and advantage of the invention to provide an herbal cleansing shampoo that includes:
about 45% to about 65% by weight water;
about 3% to about 15% by weight of an herbal extract blend component;
about 15% to about 35% by weight of a surfactant blend component;
about 3% to about 12% by weight of an oil component; and
about 1% to about 9% by weight of a performance and processing aid component;
wherein the herbal extract component includes three or more of ocmium basilicum leaf extract, citrus aurantium amara flower extract, rosmarius officinalis leaf extract, chamomilia recutia flower extract, lavandula angustifolia flower extract, artemisia vulgaris herb extract, ocmium basilicum seed extract, eugenia caryophyllus bud extract, and thymus vulgaris extract;
and the performance and processing aid component comprises two or more of citric acid, sodium benzoate, potassium sorbate, pentylene glycol, sodium anisate, aqueous trisodium ethylenediamine disuccinate, and hydroxypropyl methylcellulose.

In another embodiment, it is a feature and advantage of the invention to provide an herbal cleansing shampoo that includes:
about 50% to about 60% by weight water;
about 5% to about 10% by weight of an herbal extract blend component;
about 20% to about 30% by weight of a surfactant blend component;
about 5% to about 10% by weight of an oil component; and
about 2% to about 8% by weight of a performance and processing aid component;
wherein the herbal extract component includes five or more of ocmium basilicum leaf extract, citrus aurantium amara flower extract, rosmarius officinalis leaf extract, chamomilia recutia flower extract, lavandula angustifolia flower extract, artemisia vulgaris herb extract, ocmium basilicum seed extract, eugenia caryophyllus bud extract, and thymus vulgaris extract;
and the performance and processing aid component comprises four or more of citric acid, sodium benzoate, potassium sorbate, pentylene glycol, sodium anisate, aqueous trisodium ethylenediamine disuccinate, and hydroxypropyl methylcellulose.

Additional embodiments of the invention are provided in the following Detailed Description.

### DETAILED DESCRIPTION OF THE INVENTION

The herbal cleansing shampoo of the invention is designed to provide the user with shiner, healthier hair with a luscious appearance, and is environmentally friendly. The herbal cleansing shampoo can be used alone as a shampoo, or as part of an overall hair treatment sequence. One example of an overall hair treatment sequence can include first treating the hair with a pre-shampoo argan oil blend hair treatment as described in co-pending U.S. Patent Application ______, entitled “Argan Oil Blend Hair Treatment” and separately rinsing the hair with a rinse-free nutrient rich rinse as described in co-pending U.S. Patent Application ______, entitled “Rinse-Free Nutrient Rich Rinse.” Both co-pending applications are filed on even date herewith and are incorporated herein by reference.

In addition to water, the herbal cleansing shampoo can include an herbal extract blend component, a surfactant blend component, a natural oil component, and a performance and processing aid component. The water component is preferably deionized water and can constitute about 40% to about 70% by weight of the herbal cleansing shampoo, or about 45% to about 55% by weight, or about 50% to about 60% by weight. The herbal extract blend component can constitute about 2% to about 20% by weight of the herbal cleansing shampoo, or about 13% to about 15% by weight, or about 5% to about 10% by weight. The surfactant blend component can constitute about 10% to about 40% by weight of the herbal cleansing shampoo, or about 15% to about 35% by weight, or about 20% to about 30% by weight. The natural oil component can constitute about 1% to about 15% by weight of the herbal cleansing shampoo, or about 3% to about 12% by weight, or about 5% to about 10% by weight. The performance and processing aid component can constitute 0.5% to about 10% by weight of the herbal cleansing shampoo, or about 1% to about 9% by weight, or about 2% to about 8% by weight. As used herein, the phrase “performance and processing aid” includes ingredients that stabilize and maintain the performance of other ingredients in the herbal cleansing shampoo, ingredients that enhance the performance of other ingredients, and ingredients that aid in the manufacture, processing, and use of the herbal cleansing shampoo.

The herbal extract blend component of the herbal cleansing shampoo can include two or more, three or more, four or more, five or more, six or more, or seven or more of ocmium basilicum leaf extract, citrus aurantium amara flower extract, rosmarius officinalis leaf extract, chamomilia recutia flower extract, lavandula angustifolia flower extract, artemisia vulgaris herb extract, ocmium basilicum seed extract, eugenia caryophyllus bud extract, and thymus vulgaris extract. Each of these herbal extract ingredients can, when present, constitute about 0.1% to about 5% by weight of the herbal cleansing shampoo, or about 0.3% to about 3% by weight, or about 0.5% to about 1.5% by weight.

The surfactant blend component can include two or more, or three or more, or four or more of guar hydroxypropyltrimonium chloride, aqueous cocamidopropyl betaine, aqueous disodium laureth suphosuccinate, sodium methyl cocoyl taurate, and sodium cocoyl isethionate. The guar hydroxypropyltrimonium chloride can, when present, constitute about 0.1% to about 2.0% by weight of the herbal cleansing shampoo, or about 0.2% to about 1.0% by weight, or about 0.3% to about 5% by weight. The aqueous cocamidopropyl betaine can constitute about 2% to about 20% by weight of the herbal cleansing shampoo, or about 5% to about 15% by weight, or about 8% to about 12% by weight. The aqueous disodium laureth suphosuccinate can constitute about 3% to about 20% by weight of the herbal cleansing shampoo, or about 6% to about 17% by weight, or about 10% to about 14% by weight.

The natural oil component can include one or both of glycerin and tea tree oil (also known as melaleuca alternifolia leaf oil). The glycerin can constitute about 1% to about 15% by weight of the herbal cleansing shampoo, or about 3% to about 12% by weight, or about 5% to about 10% by weight. The tea tree oil can constitute about 0.05% to about 2% by weight of the herbal cleansing shampoo, or about 0.10% to about 1% by weight, or about 0.3% to about 0.5% by weight.

The performance and processing aid component can include two or more, three or more, four or more, five or more, or six or more of citric acid, aqueous cetrimonium chloride, sodium benzoate, potassium sorbate, pentylene glycol, sodium anisate, aqueous trisodium ethylenediamine disuccinate, and hydroxypropyl methylcellulose. Each of these ingredients can, when present, constitute about 0.05% to about 5% by weight of the herbal cleansing shampoo, or about 0.10% to about 3% by weight, or about 0.15% to about 2% by weight. Centrimonium chloride currently has a regulatory upper limit of 0.3% for leave-on products, allowing a 30% aqueous blend to be used at up to 1% by weight of the herbal cleansing shampoo.

The following Table 1 shows an example of an herbal cleansing shampoo formulation useful in the invention.

TABLE 1
Example Herbal Cleansing Shampoo Formula (% By Weight)

| | Broad | Intermediate | Narrow |
|---|---|---|---|
| Component | Range | Range | Range |
| Deionized Water | 40-70 | 45-65 | 50-60 |
| Herbal Extract Blend Component | 2-20 | 3-15 | 5-10 |
| ocmium basilicum leaf extract | 0.1-5 | 0.3-3 | 0.5-1.5 |
| citrus aurantium amara flower extract | 0.1-5 | 0.3-3 | 0.5-1.5 |
| rosmarius officinalis leaf extract | 0.1-5 | 0.3-3 | 0.5-1.5 |
| chamomilia recutia flower extract | 0.1-5 | 0.3-3 | 0.5-1.5 |
| lavandula angustifolia flower extract | 0.1-5 | 0.3-3 | 0.5-1.5 |
| artemisia vulgaris herb extract | 0.1-5 | 0.3-3 | 0.5-1.5 |
| ocmium basilicum seed extract | 0.1-5 | 0.3-3 | 0.5-1.5 |
| eugenia caryophyllus bud extract | 0.1-5 | 0.3-3 | 0.5-1.5 |
| thymus vulgaris extract | 0.1-5 | 0.3-3 | 0.5-1.5 |
| Surfactant Blend Component | 10-40 | 15-35 | 20-30 |
| guar hydroxypropyltrimonium chloride | 0.1-2 | 0.2-1 | 0.3-0.5 |
| aqueous cocamidopropyl betaine (30%) | 2-20 | 5-15 | 8-12 |
| aqueous disodium laureth suphosuccinate | 3-20 | 6-17 | 10-14 |
| (30%) | | | |
| sodium methyl cocoyl taurate (95%) | 0.5-5 | 1-3 | 1.5-2 |
| sodium cocoyl isethionate (85%) | 1-10 | 2-8 | 3-6 |
| Natural Oil Component | 1-15 | 3-12 | 5-10 |
| glycerin | 1-15 | 3-12 | 5-10 |
| tea tree oil | 0.05-2 | 0.10-1 | 0.15-0.5 |
| Performance and Processing Aid Component | 0.5-10 | 1-9 | 2-8 |
| citric acid | 0.05-5 | 0.10-3 | 0.15-2 |
| aqueous cetrimonium chloride (30%) | 0.05-5 | 0.10-3 | 0.15-1* |
| sodium benzoate | 0.05-5 | 0.10-3 | 0.15-2 |
| potassium sorbate | 0.05-5 | 0.10-3 | 0.15-2 |
| pentylene glycol | 0.05-5 | 0.10-3 | 0.15-2 |
| sodium anisate | 0.05-5 | 0.10-3 | 0.15-2 |
| aqueous trisodium ethylenediamine | 0.05-5 | 0.10-3 | 0.15-2 |
| disuccinate | | | |
| hydroxypropyl methylcellulose | 0.05-5 | 0.10-3 | 0.15-2 |
| TOTAL | 100 | 100 | 100 |

*Centrimoium chloride currently has a regulatory upper limit of 0.3% excluding water.

The ingredients of the herbal cleansing shampoo can be added and mixed in any suitable order, using any suitable mixer. In one embodiment, the glycerin, hydroxypropylmethylcellulose, and guar hydroxypropyltrimonium chloride can first be mixed together as a slurry, followed by the addition of water with rapid propeller mixing. The aqueous trisodium ethylenediamine disuccinate, pentylene glycol, and sodium anisate can then be added and mixed until the polymers are fully hydrated. The aqueous cocamidopropyl betaine, aqueous disodium laureth suphosuccinate, sodium methyl cocoyl taurate, and sodium cocoyl isethionatecan then be added and mixed using heat until the surfactants are fully dissolved. The ingredients of the herbal extract blend component and the tea tree oil can then be added and mixed with colling at a temperature below 40° C. to yield the herbal cleansing shampoo.

Several ingredients of the herbal cleansing shampoo have advantageous properties that, together and alone, contribute to shiny, healthy, thicker, and younger-looking hair and a healthy scalp. Referring to the ingredients of the herbal extract blend component, ocmium basilicum leaf extract contains antioxidants that help to nourish and strengthen hair follicles, promoting healthy hair growth. It also has antibacterial properties that help to prevent scalp infections and dandruff, leaving the hair clean and healthy. Citrus aurantium amara flower extract moisturizes the hair shaft and prevents the hair from becoming brittle, dull, or scratchy. Rosmarius officinalis leaf extract is believed to help heal nerve endings and may rejuvenate nerves in the scalp, aiding hair growth. Chamomilia recutia flower extract helps promote hair strength, helps prevent split ends, and lightens hair strands. Lavandula angustifolia flower extract can strengthen hair follicles, promote hair growth, and reduce frizz. Artemisia vulgaris herb extract can strengthen hair roots, reduce hair loss, and improve scalp health. Ocmium basilicum seed extract moisturizes the hair and scalp and can reduce hair loss. Eugenia caryophyllus bud extract helps to combat dry hair and split ends and promotes hair follicle activity to encourage hair growth. Thymus vulgaris extract eases inflammation affecting the scalp and hair follicles to help combat dandruff.

Citric acid helps remove buildup of debris in hair, resulting in shinier hair, and enhances the performance of the remaining ingredients. Glycerin combats dandruff and split ends by moisturizing the scalp and strengthening and moisturizing the hair. Tea tree oil has antifungal and antibacterial properties that help to maintain a clean healthy scalp and strong, healthy hair follicles. Centrimonium chloride has antimicrobial and anti-static properties for hair.

Sodium benzoate, potassium sorbate, and sodium anisate act as preservatives in the herbal cleansing shampoo and prevent the growth of microbes and fungi. Pentylene glycol promotes blending and processing of the other shampoo ingredients and acts as a moisturizer. Aqueous trisodium ethylenediamine disuccinate binds to metal ions and metallic compounds to prevent them from adhering to the scalp or hair. Hydroxypropyl methylcellulose acts as a thickening aid for the herbal cleansing shampoo.

The herbal cleansing shampoo can be applied to the user's hair once per week or as needed, using up to six pump strokes for shoulder length hair with average thickness. Once applied, the herbal cleansing shampoo can be massaged gently into the scalp and hair and can be rinsed using warm water until the hair is clean. The user's hair can optionally be treated with a conditioner, for example using an argan oil blend hair treatment as described in the above- identified co-pending U.S. Patent Application ______, entitled “Argan Oil blend Hair Treatment” and, after shampooing, can be rinsed using a rinse-free nutrient rich rinse as described in co-pending U.S. Patent Application ______, entitled “Rinse-Free Nutrient Rich Rinse,” the disclosures of which are incorporated herein by reference.

The embodiments of the invention described herein are exemplary. Various modifications and improvements can be made without departing from the spirit and scope of the invention. The scope of the invention is defined in the appended claims, and all changes that fall within the meaning and range of equivalency are intended to be embraced therein.

1. An herbal cleansing shampoo, comprising:
about 40% to about 70% by weight water;
about 2% to about 20% by weight of an herbal extract blend component;
about 10% to about 40% by weight of a surfactant blend component; and
about 1% to about 15% by weight of a natural oil component;
wherein the herbal extract component includes three or more of ocmium basilicum leaf extract, citrus aurantium amara flower extract, rosmarius officinalis leaf extract, chamomilia recutia flower extract, lavandula angustifolia flower extract, artemisia vulgaris herb extract, ocmium basilicum seed extract, eugenia caryophyllus bud extract, and thymus vulgaris extract.

2. The herbal cleansing shampoo of claim 1, wherein the herbal extract blend component comprises five or more of ocmium basilicum leaf extract, citrus aurantium amara flower extract, rosmarius officinalis leaf extract, chamomilia recutia flower extract, lavandula angustifolia flower extract, artemisia vulgaris herb extract, ocmium basilicum seed extract, eugenia caryophyllus bud extract, and thymus vulgaris extract.

3. The herbal cleansing shampoo of claim 1, wherein the herbal extract blend component comprises seven or more of ocmium basilicum leaf extract, citrus aurantium amara flower extract, rosmarius officinalis leaf extract, chamomilia recutia flower extract, lavandula angustifolia flower extract, artemisia vulgaris herb extract, ocmium basilicum seed extract, eugenia caryophyllus bud extract, and thymus vulgaris extract.

4. The herbal cleansing shampoo of claim 1, wherein the surfactant blend component comprises two or more of guar hydroxypropyltrimonium chloride, cetrimonium chloride, aqueous cocamidopropyl betaine, aqueous disodium laureth suphosuccinate, sodium methyl cocoyl taurate, and sodium cocoyl isethionate.

5. The herbal cleansing shampoo of claim 1, wherein the surfactant blend component comprises three or more of guar hydroxypropyltrimonium chloride, cetrimonium chloride, aqueous cocamidopropyl betaine, aqueous disodium laureth suphosuccinate, sodium methyl cocoyl taurate, and sodium cocoyl isethionate.

6. The herbal cleansing shampoo of claim 1, wherein the surfactant blend component comprises four or more of guar hydroxypropyltrimonium chloride, cetrimonium chloride, aqueous cocamidopropyl betaine, aqueous disodium laureth suphosuccinate, sodium methyl cocoyl taurate, and sodium cocoyl isethionate.

7. The herbal cleansing shampoo of claim 1, wherein the natural oil component comprises glycerin.

8. The herbal cleansing shampoo of claim 7, wherein the natural oil component further comprises tea tree oil.

9. The herbal cleansing shampoo of claim 1, further comprising a performance and processing aid component, the performance and processing aid component including two or more of citric acid, sodium benzoate, potassium sorbate, pentylene glycol, sodium anisate, aqueous trisodium ethylenediamine disuccinate, and hydroxypropyl methylcellulose.

10. The herbal cleansing shampoo of claim 1, comprising:
about 45% to about 65% by weight of the water;
about 3% to about 15% by weight of the herbal extract blend component;
about 15% to about 35% by weight of the surfactant blend component; and
about 3% to about 12% by weight of the natural oil component.

11. The herbal cleansing shampoo of claim 1, comprising:
about 50% to about 60% by weight of the water;
about 5% to about 10% by weight of the herbal extract blend component;
about 20% to about 30% by weight of the surfactant blend component; and
about 5% to about 10% by weight of the natural oil component.

12. An herbal cleansing shampoo, comprising:
about 45% to about 65% by weight water;
about 3% to about 15% by weight of an herbal extract blend component;
about 15% to about 35% by weight of a surfactant blend component;
about 3% to about 12% by weight of an oil component; and
about 1% to about 9% by weight of a performance and processing aid component;
wherein the herbal extract component includes three or more of ocmium basilicum leaf extract, citrus aurantium amara flower extract, rosmarius officinalis leaf extract, chamomilia recutia flower extract, lavandula angustifolia flower extract, artemisia vulgaris herb extract, ocmium basilicum seed extract, eugenia caryophyllus bud extract, and thymus vulgaris extract;
and the performance and processing aid component comprises two or more of citric acid, sodium benzoate, potassium sorbate, pentylene glycol, sodium anisate, aqueous trisodium ethylenediamine disuccinate, and hydroxypropyl methylcellulose.

13. The herbal cleansing shampoo of claim 12, wherein each ingredient of the herbal extract blend component constitutes about 0.1% to about 5% by weight of the herbal cleansing shampoo.

14. The herbal cleansing shampoo of claim 12, wherein each ingredient of the herbal extract blend component constitutes about 0.3% to about 3% by weight of the herbal cleansing shampoo.

15. The herbal cleansing shampoo of claim 12, wherein each ingredient of the herbal extract blend component constitutes about 0.5% to about 1.5% by weight of the herbal cleansing shampoo.

16. The herbal cleansing shampoo of claim 12, wherein the oil component comprises glycerin.

17. The herbal cleansing shampoo of claim 16, wherein the oil component further comprises tea tree oil.

18. An herbal cleansing shampoo, comprising:
about 50% to about 60% by weight water;
about 5% to about 10% by weight of an herbal extract blend component;
about 20% to about 30% by weight of a surfactant blend component;
about 5% to about 10% by weight of a natural oil component; and
about 2% to about 8% by weight of a performance and processing aid component;
wherein the herbal extract component includes five or more of ocmium basilicum leaf extract, citrus aurantium amara flower extract, rosmarius officinalis leaf extract, chamomilia recutia flower extract, lavandula angustifolia flower extract, artemisia vulgaris herb extract, ocmium basilicum seed extract, eugenia caryophyllus bud extract, and thymus vulgaris extract;
and the performance and processing aid component comprises four or more of citric acid, sodium benzoate, potassium sorbate, pentylene glycol, sodium anisate, aqueous trisodium ethylenediamine disuccinate, and hydroxypropyl methylcellulose.

19. The herbal cleansing shampoo of claim 18, wherein the herbal extract component comprises seven or more of ocmium basilicum leaf extract, citrus aurantium amara flower extract, rosmarius officinalis leaf extract, chamomilia recutia flower extract, lavandula angustifolia flower extract, artemisia vulgaris herb extract, ocmium basilicum seed extract, eugenia caryophyllus bud extract, and thymus vulgaris extract.

20. The herbal cleansing shampoo of claim 18, wherein the performance and processing aid component comprises six or more of citric acid, sodium benzoate, potassium sorbate, pentylene glycol, sodium anisate, aqueous trisodium ethylenediamine disuccinate, and hydroxypropyl methylcellulose.
